(19) United States
(12) Patent Application Publication
(10) Pub. No.: US 2016/0303050 A1
(43) Pub. Date: Oct. 20, 2016

(54) FORMULATIONS CONTAINING DIACEREIN AND METHODS OF LOWERING BLOOD LEVELS OF URIC ACID USING THE SAME

(71) Applicant: TWi Biotechnology, Inc., Taipei (TW)

(72) Inventors: Hanpin Lim, Taipei City (TW); Carl Oscar Brown, III, San Diego, CA (US); Wei-Shu Lu, New Taipei City (TW); Tien-Kuen Chung, Taipei (TW); Chih-Kuang Chen, Taipei City (TW)

(21) Appl. No.: 14/691,293

(22) Filed: Apr. 20, 2015

(51) Int. Cl. A61K 9/24; A61K 9/20; A61K 31/222

(52) U.S. Cl. CPC A61K 9/209; A61K 31/222; A61K 9/2054

(57) ABSTRACT

A controlled-release formulation containing diacerein or its analogs is provided. Also provided is a method of lowering blood levels of uric acid using this formulation.

### CROSS-REFERENCES TO RELATED APPLICATIONS

Not applicable.

### BACKGROUND OF THE INVENTION

1. Field of the Invention

The present invention relates to a diacerein formulation, especially to a method of lowering blood levels of uric acid using this formulation.

2. Descriptions of the Related Art

Chemically, rhein is 9,10-dihydro-4,5-dihydroxy-9,10-dioxo-2-anthracene carboxylic acid having a structure of Formula (I), and one of its prodrugs, diacerein, is 4,5-bis (acetyloxy) 9,10-dihydro-4,5-dihydroxy-9,10-dioxo-2-anthracenecarboxylic acid having a structure of Formula (II). Diacerein is entirely converted into rhein before reaching the systemic circulation, and exerts its physiological function in form of rhein within the body.

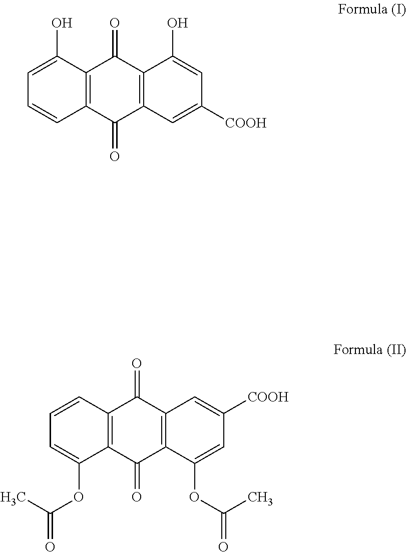 [SMILES: O=C(O)c1cc(O)c2c(c1)C(=O)c1cccc(O)c1C2=O ; CC(=O)Oc1cccc2c1C(=O)c1c(OC(C)=O)cc(C(=O)O)cc1C2=O]

Diacerein is an anti-inflammatory agent widely used in the treatment of osteoarthritis, which has been demonstrated to inhibit interleukin-1 (IL-1) signaling. Presently, diacerein capsules are available in 50 mg strength and are marketed under various trade names in different countries, including Art 50®, Artrodar®, etc. As disclosed in U.S. Pat. No. 8,536,152, diacerein can also be used as an adjunctive treatment for type II diabetes mellitus. Although diacerein can be administered by oral route, it cannot be completely absorbed by the digestive tract, and the oral bioavailability of diacerein has been estimated to be approximately 40% to 60%. The incomplete absorption of diacerein may result in undesirable side effects such as diarrhea or soft stools. In vitro and in vivo studies have showed that non-absorbed diacerein is metabolized to rhein in the colon, which then induces a laxative effect. Thus, there is still a need in the art for a diacerein formulation having reduced adverse side effects and/or higher bioavailability compared to the current commercial formulations.

As disclosed in U.S. Pat. No. 8,865,689, diacerein was found to be effective in reducing the blood uric acid levels, and can be used for treating hyperuricemia or a metabolic disorder associated with hyperuricemia. However, no diacerein formulations specific for lowering the blood uric acid levels have been developed so far.

In view of the above demand, the present invention provides a diacerein formulation having improved properties, as well as its uses in treating diseases including, but not limited to, hyperuricemia, a metabolic disorder associated with hyperuricemia, osteoarthritis and type 2 diabetes mellitus.

### SUMMARY OF THE INVENTION

In one embodiment, the invention provides a controlled-release formulation with reduced adverse side effects and/or higher bioavailability, comprising an immediate-release layer and an extended-release layer.

In another embodiment, this invention provides a method of lowering blood levels of uric acid in a subject, comprising administering the above controlled-release formulation to the subject in need thereof.

In another embodiment, this invention provides a method of lowering blood levels of uric acid in a subject, comprising administering to the subject in need thereof a formulation containing a therapeutically effective amount of a compound selected from the group consisting of diacerein, rhein, monoacetylrhein, a prodrug and a pharmaceutically acceptable salt thereof, wherein when the formulation is administered to said subject it provides at least one of the following pharmacokinetic parameters: (i) a maximum plasma concentration C. of rhein above 5.0 μg/ml; (ii) an area under the concentration time curve AUC₀₋₄ or AUC_{0-∞} of rhein above 35.0 μg·hr/ml; (iii) Tₘₐₓ of about 3 to 4.5 hours after oral administration to the subject under a fed condition; and (iv) a plasma concentration of rhein above 2.8 μg/ml for at least 4 hours.

In another embodiment, this invention provides a method of lowering blood levels of uric acid in a subject, comprising administering to the subject in need thereof a formulation containing at least about 75 mg of a compound selected from the group consisting of diacerein, rhein, monoacetylrhein, a prodrug and a pharmaceutically acceptable salt thereof.

The detailed technology and preferred embodiments implemented for the subject invention are described in the following paragraphs accompanying the appended drawings for people skilled in this field to well appreciate the features of the claimed invention.

### BRIEF DESCRIPTION OF THE DRAWINGS

FIG. 1 shows the dissolution profiles of the controlled-release formulations A and F of the present invention, measured by the United States Pharmacopeia (USP) Apparatus II (Paddle) at 50 rpm in 900 ml of pH 6.8 PBS at 37° C.;

FIG. 2 is a statistical bar graph showing inhibition of uric acid uptake by different doses of rhein;

FIG. 3 shows the average plasma concentration-time profiles of rhein after subjects received treatment with different diacerein formulations; and

FIG. 4 is a statistical bar graph showing the serum uric acid concentrations before and after the treatment with different diacerein formulations.

### DETAILED DESCRIPTION OF THE INVENTION

The term “Immediate-Release” or “IR,” as used herein, means that a drug (e.g., diacerein) is released in a conventional or non-modified way.

The term “Controlled-Release” or “CR” and “Extended-Release” or “ER,” as used herein, refers to the gradual release of a drug at a predetermined rate other than an immediate release manner over a period of time.

The term “therapeutically effective amount,” as used herein, refers to an amount that alleviates or reduces one or more symptoms of a disease.

The term “Cₘₐₓ,” as used herein, refers to the maximum observed plasma concentration, calculated as the mean of the individual maximum blood plasma concentrations.

The term “average plasma concentration,” as used herein, refers to the arithmetic mean blood plasma concentration.

The term “Tₘₐₓ,” as used herein, refers to the time at which the peak (maximum) observed blood plasma drug concentration for each individual participating in the bioavailability study.

The term “AUC_{0-∞}” or “AUC_{inf},” as used herein, refers to the mean area under the plasma/serum/blood concentration-time curve extrapolated to infinity. It is calculated as the arithmetic mean of the area under the plasma concentration-time curve from time zero extrapolated to infinity, calculated for each individual participating in the bioavailability study.

The term “AUC₀₋ₜ,” as used herein, refers to the area under the plasma/serum/blood concentration-time curve from time zero to time t, where “t” is the last sampling time point with measurable concentration for individual formulation.

The term “diacerein or its analogs,” as used herein, refers to diacerein, rhein, monoacetylrhein, or a pharmaceutically acceptable salt or a prodrug thereof.

Unless otherwise stated herein, the terms “a (an)”, “the” or the like used in this specification (especially in the Claims hereinafter) shall be understood to encompass both the singular form and the plural form.

As stated above, to improve adverse side effects and/or bioavailability of diacerein, the present invention provides a controlled-release formulation, comprising an immediate-release layer and an extended-release layer.

In one embodiment, the immediate-release layer comprises a therapeutically effective amount of a compound selected from the group consisting of diacerein, rhein, monoacetylrhein, a prodrug and a pharmaceutically acceptable salt thereof (hereinafter referred to as “diacerein or its analogs”); a filler; a binder; a disintegrant; and a lubricant; and the extended-release layer comprises a therapeutically effective amount of diacerein or its analogs, a controlled-release polymer, a filler, and a lubricant; and wherein the weight ratio of diacerein or its analogs in the immediate-release layer to that in the extended-release layer is about 2:1 to about 1:9.

In one embodiment, the formulation further comprises a cosmetic coating.

Preferably, in the formulation of the present invention, the immediate-release layer comprises about 5% to about 60% by weight, preferably about 5% to about 50% by weight of diacerein or its analogs; about 30% to about 95% by weight, preferably about 40% to about 85% by weight of a filler; about 0.1% to about 20% by weight, preferably about 1% to about 10% by weight of a binder; about 0.1% to about 20% by weight, preferably about 1% to about 10% by weight of a disintegrant; and about 0.01% to about 5% by weight, preferably about 0.1% to about 2.5% by weight of a lubricant, based on the total weight of the immediate-release layer; and the extended-release layer comprises about 5% to about 60% by weight, preferably about 5% to about 50% by weight of diacerein or its analogs; about 1% to about 60% by weight, preferably about 10% to about 50% by weight of a controlled-release polymer; about 1% to about 70% by weight, preferably about 10% to about 55% by weight of a filler; and about 0.01% to about 5% by weight, preferably about 0.1% to about 2.5% by weight of a lubricant, based on the total weight of the extended-release layer.

Examples of fillers include, but are not limited to, lactose monohydrate, lactose anhydrous, and starches. Preferably, the filler is lactose monohydrate.

Examples of binders include, but are not limited to, povidone, starch, gelatin, tragacanth, methylcellulose, hypromellose, and hydroxypropylcellulose. Preferably, the binder is povidone.

Suitable disintegrants include, but are not limited to, sodium carboxymethylcellulose, L-hydroxypropylcellulose, cropovidone, corn starch, sodium starch glycolate, starch, croscarmellose sodium, and alginic acid or its sodium salt. Preferably, the disintegrant is croscarmellose sodium.

Suitable lubricants include, but are not limited to, light anhydrous silicic acid, talc, stearic acid and its zinc, magnesium, or calcium salt, and polyethyleneglycol. Preferably, the lubricant is magnesium stearate.

Controlled-release polymers that can be used in the present invention may be, for instance, hydroxypropyl methylcellulose (HPMC), hydroxypropyl cellulose, sodium alginate, carbomer, sodium carboxymethyl cellulose, xanthan gum, guar gum, locust bean gum, poly vinyl acetate, polyvinyl alcohol carboxyvinyl polymers, polyvinyl alcohols, glucans, scleroglucans, mannans, xanthans, alginic acid and its derivatives, polyanhydrides, polyaminoacids, carboxymethyl cellulose, cross-linked sodium carboxymethyl cellulose, polyvinyl pyrrolidone, cross-linked polyvinyl pyrrolidone, carboxymethylamide, potassium methacrylate/divinylbenzene copolymer, starches and their derivatives, P-cyclodextrin, dextrin derivatives with linear or branched chains, ethyl cellulose, methyl cellulose, methacrylic acid copolymers, and cellulose derivatives. Preferably, the controlled-release polymer is hydroxypropyl methylcellulose (HPMC).

Because the formulation of the invention has reduced adverse side effects, it can deliver a higher dose of diacerein without increasing the side effects like diarrhea. Specifically, it can be administered to patients with a higher dose compared to the commercial diacerein drugs (e.g., Artrodar®, 50 mg Q.D. or B.I.D, 50 or 100 mg daily in total), and may contain at least about 75 mg, preferably about 75 to 200 mg, more preferably about 75 to 100 mg of diacerein or its analogs, thereby enhancing the treatment effect in a single dose.

In another aspect, the inventors of the present application found that the formulation containing at least about 75 mg of diacerein is more effective in reducing blood levels of uric acid than Artrodar® (an immediate-release formulation containing 50 mg of diacerein). Thus, the formulation of the invention comprises preferably at least about 75 mg, more preferably about 75 to 200 mg, most preferably about 75 to 100 mg of diacerein or its analogs.

In a dissolution test, the controlled-release formulation preferably has an in vitro dissolution rate when measured by the United States Pharmacopeia (USP) Apparatus II (Paddle) at 50 rpm in 900 ml of pH 6.8 PBS at 37° C., between about 30% and about 45%, preferably about 35% and about 40%, diacerein released after 1 hour; between about 50% and about 60% diacerein released after 4 hours; between about 60% and about 75%, preferably about 65% and about 75%, diacerein released after 8 hours; and not less than about 80% diacerein released after 16 hours, by weight.

In one embodiment, the controlled-release formulation of the invention, when administered to a subject, may provide at least one of the following pharmacokinetic parameters: (i) a maximum plasma concentration Cₘₐₓ of rhein above 5.0 μg/ml; (ii) an area under the concentration time curve AUC₀₋ₜ or AUC_{0-∞} of rhein above 35.0 μg·hr/ml; (iii) Tₘₐₓ of about 3 to 4.5 hours after oral administration to a subject under a fed condition; and (iv) a plasma concentration of rhein above 2.8 μg/ml for at least 4 hours. A formulation exhibiting the above pharmacokinetic parameters shows reduced adverse side effects, reduced food effect, higher bioavailability, and/or better effect in reducing the blood uric acid levels as compared to the conventional immediate-release formulation.

Preferably, the formulation is a once-daily (i.e., taken once per day) controlled-release formulation.

Because the formulation of the invention has the above-mentioned advantages, it is beneficial when used for treating all the diseases to which diacerein is therapeutically effective. These diseases include, but are not limited to, hyperuricemia, a metabolic disorder associated with hyperuricemia, osteoarthritis and type 2 diabetes mellitus. The metabolic disorder associated with hyperuricemia includes, but is not limited to, acute gout, chronic gout, gout arthritis, gout flares, uric acid nephrolithiasis, gouty nephropathy, cardiovascular diseases (e.g., hypertension and atherosclerosis), obesity, chronic kidney disease, and insulin resistance.

The formulation can be used for decreasing inflammatory effects of gout arthritis and gout flares induced by hyperuricemia; and/or dissolving kidney stones; and/or reducing the recurrence rate of acute inflammatory arthritis induced by hyperuricemia; and/or slowing down the progression of urate nephropathy in a subject.

In one embodiment, the formulation may further comprise one or more additional therapeutic agent, such as an anti-inflammatory agent or a urate-lowering agent to enhance the therapeutic effect of diacerein. Examples of the anti-inflammatory agents include, but are not limited to, non-steroidal anti-inflammatory drugs (NSAIDs), corticosteroids, and colchicines. Examples of the urate-lowering agents include, but are not limited to, xanthine oxidase inhibitors, uricosuric agents, urate oxidases, urinary alkalinizers, and fenofibrate.

The present invention also provides a method of lowering blood levels of uric acid in a subject, comprising administering to the subject in need thereof a formulation containing diacerein or its analogs.

The formulations that can be used in this method may have the structure, composition and other properties as those defined above for the formulation of the present invention. Alternatively, the formulations suitable for this method may have different structure and composition as long as when administered to a subject they can provide at least one of the following pharmacokinetic parameters: (i) a maximum plasma concentration Cₘₐₓ of rhein above 5.0 μg/ml; (ii) an area under the concentration time curve AUC₀₋ₜ or AUC_{0-∞} of rhein above 35.0 μg·hr/ml; (iii) Tₘₐₓ of about 3 to 4.5 hours after oral administration to the subject under a fed condition; and (iv) a plasma concentration of rhein above 2.8 μg/ml for at least 4 hours.

In another embodiment, the formulation used in the method contains at least about 75 mg, preferably about 75 to 200 mg, more preferably about 75 to 100 mg of diacerein or its analogs.

Hereinafter, the present invention will be further illustrated with reference to the following examples. However, these examples are only provided for illustrate purpose, but not to limit the scope of the present invention.

### PREPARATION EXAMPLE

### Preparation of a Controlled-Release Formulation Containing Diacerein

Ten controlled-release tablet formulations containing 75 or 100 mg of diacerein were prepared according to Tables 1(a) and 1(b). The prepared tablets were used in the following in vivo study.

TABLE 1(a)

| | | 75 mg diacerein | | | | | | | | | |
|---|---|---|---|---|---|---|---|---|---|---|---|
| | | Tablet A | | Tablet B | | Tablet C | | Tablet D | | Tablet E | |
| | | mg/ | % | mg/ | % | mg/ | % | mg/ | % | mg/ | % |
| | Ingredients | tab | w/w | tab | w/w | tab | w/w | tab | w/w | tab | w/w |
| IR Layer | Diacerein | 25 | 25 | 25 | 25 | 37.5 | 37.5 | 7.5 | 7.5 | 7.5 | 7.5 |
| | Lactose | 63.5 | 63.5 | 63.5 | 63.5 | 53 | 53 | 83 | 83 | 85 | 85 |
| | Povidone | 5 | 5 | 5 | 5 | 5 | 5 | 5 | 5 | 5 | 5 |
| | Croscarmellose Sodium | 6 | 6 | 6 | 6 | 4 | 4 | 4 | 4 | 2 | 2 |
| | Magnesium Stearate | 0.5 | 0.5 | 0.5 | 0.5 | 0.5 | 0.5 | 0.5 | 0.5 | 0.5 | 0.5 |
| | Sub Total | 100 | 100 | 100 | 100 | 100 | 100 | 100 | 100 | 100 | 100 |
| ER Layer | Diacerein | 50 | 29.7 | 50 | 29.7 | 37.5 | 22.28 | 67.5 | 40.1 | 67.5 | 40.1 |
| | Hypromellose (HPMC) | 33.5 | 19.9 | 67.34 | 40 | 50.5 | 30 | 16.83 | 10 | 67.34 | 40 |
| | Lactose | 83.83 | 49.8 | 49.99 | 29.7 | 79.33 | 47.12 | 83 | 49.3 | 32.49 | 19.3 |
| | FD&C Blue Aluminum | 0.17 | 0.1 | 0.17 | 0.1 | 0.17 | 0.1 | 0.17 | 0.1 | 0.17 | 0.1 |
| | Lake | | | | | | | | | | |
| | Magnesium Stearate | 0.84 | 0.5 | 0.84 | 0.5 | 0.84 | 0.5 | 0.84 | 0.5 | 0.84 | 0.5 |
| | Sub Total | 168.34 | 100 | 168.34 | 100 | 168.34 | 100 | 168.34 | 100 | 168.34 | 100 |
| Total Core Tablet Weight | | 268.34 | | 268.34 | | 268 34 | | 268.34 | | 268.34 | |
| Cosmetic | Opadry II Yellow | 8.3 | 3 | 8.3 | 3 | 8.3 | 3 | 8.3 | 3 | 8.3 | 3 |
| Coating | | | | | | | | | | | |

| | | | | | | | | | | |
|---|---|---|---|---|---|---|---|---|---|---|
| Total Coated Tablet Weight | 276.64 | 100 | 276.64 | 100 | 276.64 | 100 | 276.64 | 100 | 276.64 | 100 |

TABLE 1(b)
100 mg diacerein

| | | Tablet F | | Tablet G | | Tablet H | | Tablet I | | Tablet J | |
|---|---|---|---|---|---|---|---|---|---|---|---|
| | | mg/ | % | mg/ | % | mg/ | % | mg/ | % | mg/ | % |
| | Ingredients | tab | w/w | tab | w/w | tab | w/w | tab | w/w | tab | w/w |
| IR Layer | Diacerein | 33.33 | 25 | 33.33 | 25 | 50 | 37.5 | 10 | 7.5 | 10 | 7.5 |
| | Lactose | 84.67 | 63.5 | 84.66 | 63.49 | 70.66 | 52.99 | 110.66 | 82.99 | 113.32 | 84.99 |
| | Povidone | 6.67 | 5 | 6.67 | 5 | 6.67 | 5 | 6.67 | 5 | 6.67 | 5 |
| | Croscarmellose Sodium | 8 | 6 | 8 | 6 | 5.33 | 4 | 5.33 | 4 | 2.67 | 2 |
| | Magnesium Stearate | 0.67 | 0.5 | 0.67 | 0.5 | 0.67 | 0.5 | 0.67 | 0.5 | 0.67 | 0.5 |
| | Sub Total | 133.34 | 100 | 133.33 | 100 | 133.33 | 100 | 133.33 | 100 | 133.33 | 100 |
| ER Layer | Diacerein | 66.67 | 29.7 | 66.67 | 29.7 | 50 | 22.28 | 90 | 40.1 | 90 | 40.1 |
| | Hypromellose (HPMC) | 44.67 | 19.9 | 89.78 | 40 | 67.34 | 30 | 22.45 | 10 | 89.78 | 40 |
| | Lactose | 111.77 | 49.8 | 66.65 | 29.7 | 105.77 | 47.12 | 110.66 | 49.3 | 43.32 | 19.3 |
| | FD&C Blue Aluminum | 0.23 | 0.1 | 0.23 | 0.1 | 0.23 | 0.1 | 0.23 | 0.1 | 0.23 | 0.1 |
| | Lake | | | | | | | | | | |
| | Magnesium Stearate | 1.12 | 0.5 | 1.12 | 0.5 | 1.12 | 0.5 | 1.12 | 0.5 | 1.12 | 0.5 |
| | Sub Total | 224.45 | 100 | 224.45 | 100 | 224.45 | 100 | 224.45 | 100 | 224.45 | 100 |
| Total Core Tablet Weight | | 357.79 | | 357.78 | | 357.78 | | 357.78 | | 357.78 | |
| Cosmetic | Opadry II Red | 11.05 | 3 | 11.05 | 3 | 11.05 | 3 | 11.05 | 3 | 11.05 | 3 |
| Coating | | | | | | | | | | | |

| | | | | | | | | | | |
|---|---|---|---|---|---|---|---|---|---|---|
| Total Coated Tablet Weight | 368.84 | 100 | 368.83 | 100 | 368.83 | 100 | 368.83 | 100 | 368.83 | 100 |

### EXAMPLE 1

### Dissolution Assay for Diacerein Controlled-Release Formulations

In this example, dissolution was performed in accordance with the USP Apparatus II (Paddle). A solution of pH 6.8 PBS was used as the dissolution medium. Samples were taken at suitable time intervals and analyzed for diacerein content by means of high-pressure liquid chromatography (HPLC).

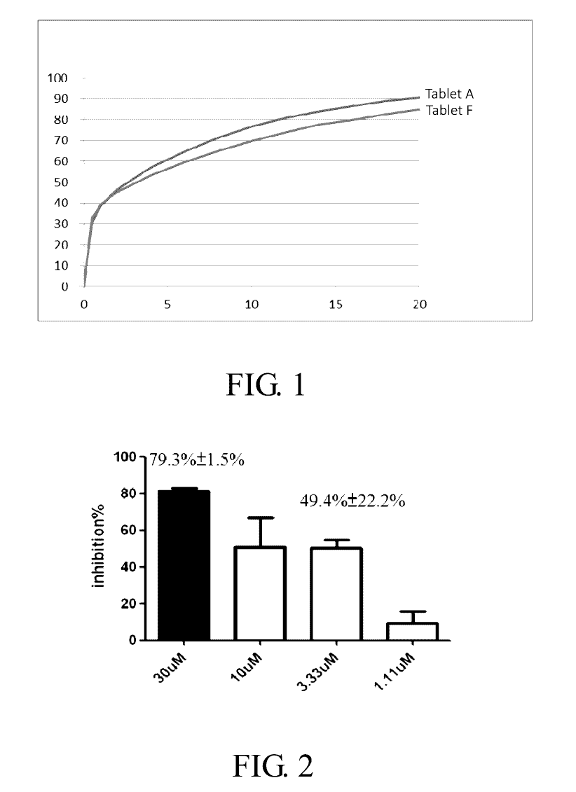

Table 2 summarizes the raw data of the dissolution of Tablets A and F of the present invention, and FIG. 1 shows the dissolution profiles.

TABLE 2

| Time | Tablet A | Tablet F |
|---|---|---|
| (hrs) | (% released) | (% released) |
| 0 | 0 | 0 |
| 0.5 | 31 | 33 |
| 1 | 39 | 40 |
| 2 | 47 | 46 |
| 4 | 57 | 53 |
| 6 | 65 | 60 |
| 8 | 71 | 65 |
| 10 | 77 | 70 |
| 12 | 81 | 74 |
| 14 | 84 | 78 |
| 16 | 87 | 80 |
| 18 | 89 | 83 |
| 20 | 91 | 85 |

Dissolution method: USP Apparatus II (Paddle), 50 rpm/900 mL pH 6.8 PBS, 37° C.

### EXAMPLE 2

### Human URAT1 Dependent Uric Acid Uptake Assay

Uric acid is mainly eliminated through urinary excretion and up to 90% of filtered urate is re-absorbed. A decrease in an excretion rate of urate is considered to elevate serum uric acid, resulting in hyperuricemia. URAT1 (urate transporter 1, the SLC22A12 gene) is the main transporter responsible for tubular reabsorption of urate and is thought to be the major mechanism for regulating blood urate levels. URAT1 has been genetically associated with urate levels, and inhibition of URAT1 may decrease serum uric acid.

In this study, an in vitro method was established to investigate the hURAT1-mediated uric acid [⁸⁻¹⁴C] uptake in transiently transfected HEK293T cells, a human embryonic kidney 293 cells containing the URAT1 transporter.

After 24 h to 72 h incubation of the transfected HEK293T cells, they were reseeded in a microplate. At least 12 hours after the cell plated, the culture medium was removed and the cells were washed and then incubated in a 100 μl Cl-free HBSS Buffer for 5 to 10 mins. The buffer was removed and 50 μl per well of Cl-free HBSS Buffer containing 50 μM uric acid [⁸⁻¹⁴] (0.13 μCi/well) was added with or without rhein (under four doses, 30, 10, 3.3 and 1.1 μM) to the cells incubated for 5 mins at 37° C. At the end of the incubation, uric acid [⁸⁻¹⁴C] uptake was stopped. Cells were washed three times and 50 ul per well 100 mM NaOH was added to lyse cells, which were then agitated at 600 rpm for at least 20 mins. The cells lysate was collected and 200 ul per well UltimaGold™ XR scintillation was added, and the mixture was agitated at 600 rpm for 10 mins. Finally, the microplate was counted. The results are shown in FIG. 2.

FIG. 2 shows that the uptake of uric acid was inhibited by rhein at 3.3 and 10 uM for 49.4%±22.2%, and at 30 uM for 79.3%±1.5%. IC₅₀ of rhein in URAT1 inhibition is 10 uM, which is about 2.8 ug/ml. Maintenance of plasma rhein concentration exceed 2.8 ug/ml may perform uric acid lowering effect.

This study shows that diacerein or its analogs can lower serum uric acid by inhibiting URAT1, and thus can be used to treat hyperuricemia and a metabolic disorder associated with hyperuricemia.

### EXAMPLE 3

### Pharmacokinetic Study

A Phase 1, randomized, open-Label, single dose, 4-Treatment, 4-sequence, 4-period, crossover, pharmacokinetic study of a diacerein immediate-release formulation (Artrodar® 50 mg Capsule) and three different doses of the diacerein controlled-release formulations of the present invention was conducted in healthy male and female volunteers under fed conditions.

Methodology: A 4-way crossover comparative pharmacokinetic study of Artrodar° 50 mg and three different doses of the controlled-release formulations (75, 100 and 200 mg) by oral administration in healthy male and female volunteers was conducted. There was a 7-day washout period separating the treatment periods.

Subjects: Healthy volunteers met all the inclusion and none of the exclusion criteria of the study.

Procedure: Diacerein administered in the different formulations and doses was compared in randomized fashion with a washout period of 7 days between periods. The subjects were randomly assigned to one of the treatment sequences as the following Table 3. The study started with a screening visit. Only eligible subjects participated in the study.

### Table 3. Study Sequence

| | Period 1 | | Period 2 | | Period 3 | | Period 4 |
|---|---|---|---|---|---|---|---|
| Sequence 1 | Treatment A | 7 days | Treatment B | 7 days | Treatment C | 7 days | Treatment D |
| Sequence 2 | Treatment B | | Treatment D | | Treatment A | | Treatment C |
| Sequence 3 | Treatment C | | Treatment A | | Treatment D | | Treatment B |
| Sequence 4 | Treatment D | | Treatment C | | Treatment B | | Treatment A |

Treatment A: 1 × Artrodar ® 50 mg Capsule;
Treatment B: 1 × 75 mg Tablet;
Treatment C: 1 × 100 mg Tablet;
Treatment D: 2 × 100 mg Tablet (200 mg).

The comparison between the different formulations and doses is based on a comparison within subjects rather than between subjects. The washout period of 7 days was estimated to be adequate in avoiding carry-over effects of the preceding treatments.

Statistical method(s) for efficacy/pharmacokinetic evaluations: AUC₀₋ₜ, AUC₀₋ₓ, Cₘₐₓ, and Tₘₐₓ for rhein in plasma of per-protocol (PP) population were determined and calculated by non-compartment methods. Analysis of Variance (ANOVA) was used for AUC₀₋ₜ, AUC_{0-∞}, Cₘₐₓ, and Tₘₐₓ. Tₘₐₓ was analyzed using an additional non-parametric test (Wilcoxon test).

Safety assessment was performed for all subjects who had been administered at least one dose of the study drug. The investigator obtained and recorded all observed adverse events (AEs) on the CRF or those voluntarily reported, including its intensity and relationship assessment with the investigational products. For all AEs, the investigator pursued and obtained information adequate to determine both the outcome of the AE and whether it met any seriousness criterion. All AEs had to be followed up until resolution or stabilization at a level acceptable to the investigator.

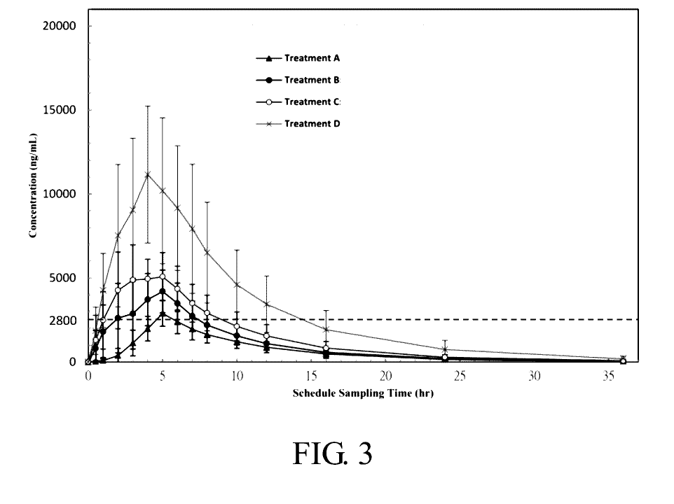

Pharmacokinetics results are shown in Tables 4 and 5 and FIG. 3. Subjects (n=23) were screened and 16 subjects were randomized into the study. There were 13 subjects who completed the whole study (4 periods) for estimating pharmacokinetics of PP population. Only the data obtained from these subjects was reported in the following tables.

TABLE 4
Pharmacokinetic parameters of rhein for PP population

| Parameter* | Treatment | Treatment | Treatment | Treatment |
|---|---|---|---|---|
| (N = 13) | A | B | C | D |
| AUC₀₋ₜ | 22,541.0 | 36,166.4 | 49,661.3 | 108,367.6 |
| (hr × ng/mL) | (4675.5) | (8983.9) | (10258.2) | (30657.3) |
| AUC_{0-∞} | 22,853.0 | 36,569.1 | 50,216.7 | 110,025.6 |
| (hr × ng/mL) | (4794.0) | (9266.3) | (10595.4) | (32067.3) |
| Cₘₐₓ | 3,018.5 | 5,360.0 | 6,483.8 | 14,590.0 |
| (ng/mL) | (663.8) | (986.3) | (1109.9) | (3116.0) |
| Tₘₐₓ | 5.00 | 3.64 | 3.62 | 4.16 |
| (hr) | (0.41) | (1.50) | (1.50) | (1.46) |

*data were shown as mean (SD)

TABLE 5
The ratio of rhein for PP population

| Ratio of treatment group | ln-transformed | | | non ln-transformed |
|---|---|---|---|---|
| (N = 13) | AUC₀₋ₜ | AUC_{0-∞} | Cₘₐₓ | Tₘₐₓ |
| Ratio of B/A (%) | 106.8 | 106.6 | 114.8 | 72.7 |
| Ratio of C/A (%) | 110.3 | 110.1 | 103.8 | 72.3 |
| Ratio of D/A (%) | 116.9 | 116.9 | 115.2 | 83.2 |

Safety results are shown in Table 6. There were no reported significant adverse events, death, or serious adverse events. During the study, the most commonly reported adverse events were diarrhea followed by nausea, vomiting, rash, blood creatine phosphokinase increased, dermatitis contact, hypotension and somnolence. Diarrhea events are almost the same between the 50 mg capsule and 75 mg and 100 mg Tablets. All adverse events were reported to be mild in intensity and were resolved in the end. In conclusion, 50 mg of Artrodar® capsule and 75, 100 and 200 mg of the Tablets are safe to use.

TABLE 6
Summary of Adverse Events (By event)

| | | Treatment | Treatment | Treatment | Treatment |
|---|---|---|---|---|---|
| | Total | A | B | C | D |
| Total AEs | 32 | 5 | 6 | 4 | 17 |
| Soft stool | 11 | 1 | 2 | 3 | 5 |
| | (34.4%) | (20.0%) | (33.3%) | (75.0%) | (29.4%) |
| Diarrhea | 8 | 1 | 2 | 1 | 4 |
| | (25.0%) | (20.0%) | (33.3%) | (25.0%) | (23.5%) |
| Nausea | 5 | 0 | 2 | 0 | 3 |
| | (15.6%) | (0.0%) | (33.3%) | (0.0%) | (17.6%) |
| Vomit | 2 | 0 | 0 | 0 | 2 |
| | (6.3%) | (0.0%) | (0.0%) | (0.0%) | (11.8%) |
| Elevated | 2 | 0 | 0 | 0 | 1 |
| CK | (6.3%) | (0.0%) | (0.0%) | (0.0%) | (5.9%) |
| Skin rash | 2 | 1 | 0 | 0 | 1 |
| | (6.3%) | (20.0%) | (0.0%) | (0.0%) | (5.9%) |
| Contact | 1 | 1 | 0 | 0 | 0 |
| dermatitis | (3.1%) | (20.0%) | (0.0%) | (0.0%) | (0.0%) |
| Sleepy | 1 | 1 | 0 | 0 | 0 |
| | (3.1%) | (20.0%) | (0.0%) | (0.0%) | (0.0%) |
| Hypotension | 1 | 0 | 0 | 0 | 1 |
| | (3.1%) | (0.0%) | (0.0%) | (0.0%) | (5.9%) |

The above results showed that the controlled-release formulations of the present invention exhibited Cₘₐₓ of rhein above 5.0 μg/ml, AUC₀₋ₜ or AUC_{0-∞} of rhein above 35.0 μg·hr/ml, and Tₘₐₓ of about 3 to 4.5 hours. In addition, the formulations of the invention provided a blood concentration of rhein above 2.8 μg/ml (the treatment effective concentration in Example 2) for at least 4 hours (Treatment B: 4.2 hours; Treatment C: 7 hours; Treatment D: 12.7 hours) and had greater bioavailability than the commercial immediate-release formulation, with about 10% greater dose-normalized AUC and Cₘₐₓ values. It was found that AUC and Cₘₐₓ values increased generally proportional with increasing doses of diacerein.

The controlled-release formulations at 75 mg and 100 mg had similar tolerability to the immediate-release formulation at 50 mg, while the 200 mg dose was associated with a higher gastrointestinal AE incidence. Therefore, compared to Artrodar® formulation, the formulations of the invention demonstrated improved safety in higher dose strengths at 75 mg and 100 mg, and provided reduced adverse side effects accordingly. This allows patients to be treated with a higher dose at 75 or 100 mg once per day without increasing the side effects.

Mean blood rhein reached peak concentrations was approximately 3.62 to 4.16 hours postdose in the tablet group of the present invention and 5.00 hours in ^{Artrodar}® capsule group under fed conditions. It has been demonstrated that fasting Tₘₐₓ of diacerein was 2.4 hours after a single oral administration of 50 mg in healthy volunteers and increased to 5.2 hours with a meal (Petitjean et al., Clinical Pharmacokinetics, November 1998, Volume 35, Issue 5, pp 347-359). The formulations of the present invention were absorbed faster under fed conditions and showed less food effect when compared to ^{Artrodar}® capsule.

### EXAMPLE 4

### Serum Uric Acid Study

Serum uric acid reduction valuation in the study of diacerein immediate-release formulation (Artrodar° 50 mg Capsule) and three different doses of the diacerein controlled-release formulations of the present invention in healthy volunteers under fed conditions was conducted.

In the pharmacokinetic study of Example 3, the effects of Artrodar° 50 mg and three different doses of the tablets of the present invention (75, 100 and 200 mg) on serum uric acid of healthy volunteers under fed conditions were also post-analyzed for intend-to-treat (ITT) population. A total of 15 subjects were analyzed. The serum uric acid concentrations were compared by before and after treatments with paired t-test analysis.

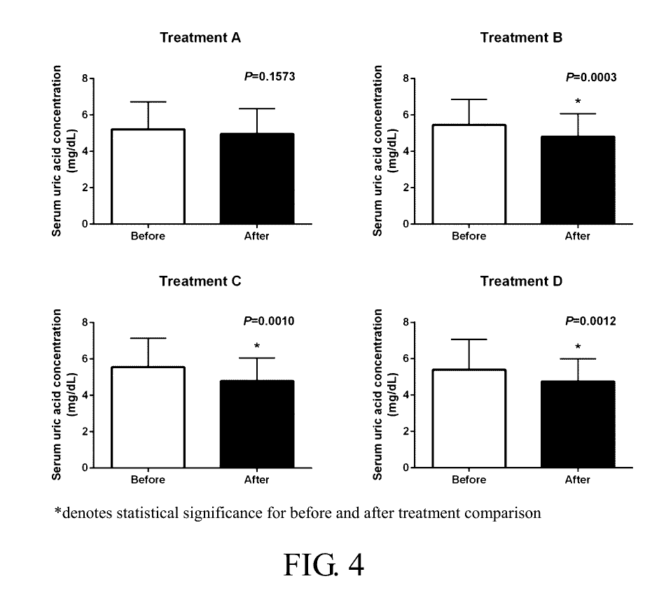

The results were revealed in FIG. 4. The serum uric acid concentration after Treatment A (Artrodar° 50 mg) was not significantly different from that before treatment.

However, the serum uric acid concentration was lowered after Treatment B than that before treatment. Same outcomes were also demonstrated in Treatment C and Treatment D. Such difference in lowering of serum uric acid might be due to the duration that rhein maintained at an effective blood concentration above 2.8 ug/ml. As shown in FIG. 3, Treatment A reached rhein blood concentration above 2.8 ug/ml for quite a short period, which was insufficient to exert urate-lowering effect.

This study revealed that serum uric acid was significantly lowered by the controlled-release formulations of the present invention at different doses above 75 mg.

The above disclosure is related to the detailed technical contents and inventive features thereof. People skilled in this field may proceed with a variety of modifications and replacements based on the disclosures and suggestions of the invention as described without departing from the characteristics thereof.

1. A method of lowering blood levels of uric acid in a subject, comprising administering to the subject in need thereof a controlled-release formulation comprising an immediate-release layer and an extended-release layer, wherein when the formulation is administered to said subject, it provides a plasma concentration of rhein above 2.8 μg/ml for at least 4 hours.

2. The method according to claim 1, wherein the immediate-release layer comprises a therapeutically effective amount of a compound selected from the group consisting of diacerein, rhein, monoacetylrhein, a prodrug and a pharmaceutically acceptable salt thereof; a filler; a binder; a disintegrant; and a lubricant; and the extended-release layer comprises a therapeutically effective amount of a compound selected from the group consisting of diacerein, rhein, monoacetylrhein, a prodrug and a pharmaceutically acceptable salt thereof; a controlled-release polymer; a filler; and a lubricant; and wherein the weight ratio of said compound in the immediate-release layer to that compound in the extended-release layer is 2:1 to 1:9.

3. The method according to claim 1, wherein the immediate-release layer comprises 5% to 60% by weight of a compound selected from the group consisting of diacerein, rhein, monoacetylrhein, a prodrug and a pharmaceutically acceptable salt thereof; 30% to 95% by weight of a filler; 0.1% to 20% by weight of a binder; 0.1% to 20% by weight of a disintegrant; and 0.01% to 5% by weight of a lubricant, based on the total weight of the immediate-release layer; and
the extended-release layer comprises 5% to 60% by weight of a compound selected from the group consisting of diacerein, rhein, monoacetylrhein, a prodrug and a pharmaceutically acceptable salt thereof; 1% to 60% by weight of a controlled-release polymer; 1% to 70% by weight of a filler; and 0.01% to 5% by weight of a lubricant, based on the total weight of the extended-release layer.

4. The method according to claim 2, wherein the controlled-release polymer is selected from the group consisting of hydroxypropyl methylcellulose (HPMC), hydroxypropyl cellulose, sodium alginate, carbomer, sodium carboxymethyl cellulose, xanthan gum, guar gum, locust bean gum, poly vinyl acetate, polyvinyl alcohol carboxyvinyl polymers, polyvinyl alcohols, glucans, scleroglucans, mannans, xanthans, alginic acid and its derivatives, polyanhydrides, polyaminoacids, carboxymethyl cellulose, cross-linked sodium carboxymethyl cellulose, polyvinyl pyrrolidone, cross-linked polyvinyl pyrrolidone, carboxymethylamide, potassium methacrylate/divinylbenzene copolymer, starches and their derivatives, β-cyclodextrin, dextrin derivatives with linear or branched chains, ethyl cellulose, methyl cellulose, methacrylic acid copolymers, cellulose derivatives, and any combinations thereof.

5. The method according to claim 1, wherein the formulation comprises at least about 75 mg of diacerein.

6. The method according to claim 1, wherein when the formulation is administered to said subject it provides at least one of the following pharmacokinetic parameters: (i) a maximum plasma concentration Cmax of rhein above 5.0 μg/ml; (ii) an area under the concentration time curve AUC0-t or AUC0-∞ of rhein above 35.0 μg·hr/ml, and (iii) Tmax of about 3 to 4.5 hours after oral administration to a subject under a fed condition.

7. The method according to claim 1, wherein the formulation is a once-daily controlled-release formulation.

8. The method according to claim 1, wherein said subject has a disease or condition selected from the group consisting of hyperuricemia, a metabolic disorder associated with hyperuricemia, osteoarthritis and type 2 diabetes mellitus.

9. A method of lowering blood levels of uric acid in a subject, comprising administering to the subject in need thereof a formulation comprising a therapeutically effective amount of a compound selected from the group consisting of diacerein, rhein, monoacetylrhein, a prodrug and a pharmaceutically acceptable salt thereof, wherein when the formulation is administered to said subject it provides at least one of the following pharmacokinetic parameters: (i) a maximum plasma concentration Cmax of rhein above 5.0 pg/ml; (ii) an area under the concentration time curve AUC0-t or AUC0-∞ of rhein above 35.0 μg·hr/ml; and (iii) Tmax of about 3 to 4.5 hours after oral administration to the subject under a fed condition.

10. The method according to claim 9, wherein the formulation is a controlled-release formulation and comprises an immediate-release layer and an extended-release layer.

11. The method according to claim 9, wherein the immediate-release layer comprises a therapeutically effective amount of a compound selected from the group consisting of diacerein, rhein, monoacetylrhein, a prodrug and a pharmaceutically acceptable salt thereof; a filler; a binder; a disintegrant; and a lubricant; and the extended-release layer comprises a therapeutically effective amount of a compound selected from the group consisting of diacerein, rhein, monoacetylrhein, a prodrug and a pharmaceutically acceptable salt thereof; a controlled-release polymer; a filler; and a lubricant; and wherein the weight ratio of said compound in the immediate-release layer to said compound in the extended-release layer is 2:1 to 1:9.

12. A method of lowering blood levels of uric acid in a subject, comprising administering to the subject in need thereof a formulation comprising at least 75 mg of a compound selected from the group consisting of diacerein, rhein, monoacetylrhein, a prodrug, and a pharmaceutically acceptable salt thereof.

13. The method according to claim 12, wherein when the formulation is administered to said subject it provides at least one of the following pharmacokinetic parameters: (i) a maximum plasma concentration Cmax of rhein above 5.0 μg/ml; (ii) an area under the concentration time curve AUC0-t or AUC0-∞ of rhein above 35.0 μg·hr/ml; and (iii) Tmax of 3 to 4.5 hours after oral administration to the subject under a fed condition.

14. The method according to claim 12, wherein the formulation is a controlled-release formulation and comprises an immediate-release layer and an extended-release layer.

15. The method according to claim 12, wherein the immediate-release layer comprises a therapeutically effective amount of a compound selected from the group consisting of diacerein, rhein, monoacetylrhein, a prodrug and a pharmaceutically acceptable salt thereof; a filler; a binder; a disintegrant; and a lubricant; and the extended-release layer comprises a therapeutically effective amount of a compound selected from the group consisting of diacerein, rhein, monoacetylrhein, a prodrug and a pharmaceutically acceptable salt thereof, a controlled-release polymer; a filler; and a lubricant; and wherein the weight ratio of said compound in the immediate-release layer to that compound in the extended-release layer is 2:1 to 1:9.

16. A controlled-release formulation with reduced adverse side effects, comprising an immediate-release layer and an extended-release layer, wherein the immediate-release layer comprises a therapeutically effective amount of a compound selected from the group consisting of diacerein, rhein, monoacetylrhein, a prodrug and a pharmaceutically acceptable salt thereof; a filler; a binder; a disintegrant; and a lubricant; and the extended-release layer comprises a compound selected from the group consisting of therapeutically effective amount of diacerein, rhein, monoacetylrhein, a prodrug and a pharmaceutically acceptable salt thereof; a controlled-release polymer; a filler; and a lubricant; and wherein the weight ratio of said compound in the immediate-release layer to that compound in the extended-release layer is about 2:1 to about 1:9.

17. The formulation according to claim 16, wherein the immediate-release layer comprises about 5% to about 60% by weight of a compound selected from the group consisting of diacerein, rhein, monoacetylrhein, a prodrug and a pharmaceutically acceptable salt thereof; about 30% to about 95% by weight of a filler; about 0.1% to about 20% by weight of a binder; about 0.1% to about 20% by weight of a disintegrant; and about 0.01% to about 5% by weight of a lubricant, based on the total weight of the immediate-release layer; and
the extended-release layer comprises about 5% to about 60% by weight of a compound selected from the group consisting of diacerein, rhein, monoacetylrhein, a prodrug and a pharmaceutically acceptable salt thereof; about 1% to about 60% by weight of a controlled-release polymer; about 1% to about 70% by weight of a filler; and about 0.01% to about 5% by weight of a lubricant, based on the total weight of the extended-release layer.

18. The formulation according to claim 16, wherein the controlled-release polymer is selected from the group consisting of hydroxypropyl methylcellulose (HPMC), hydroxypropyl cellulose, sodium alginate, carbomer, sodium carboxymethyl cellulose, xanthan gum, guar gum, locust bean gum, poly vinyl acetate, polyvinyl alcohol carboxyvinyl polymers, polyvinyl alcohols, glucans, scleroglucans, mannans, xanthans, alginic acid and its derivatives, polyanhydrides, polyaminoacids, carboxymethyl cellulose, cross-linked sodium carboxymethyl cellulose, polyvinyl pyrrolidone, cross-linked polyvinyl pyrrolidone, carboxymethylamide, potassium methacrylate/divinylbenzene copolymer, starches and their derivatives, β-cyclodextrin, dextrin derivatives with linear or branched chains, ethyl cellulose, methyl cellulose, methacrylic acid copolymers, cellulose derivatives, and any combinations thereof.

19. The formulation according to claim 16, wherein when the formulation is administered to a subject it provides at least one of the following pharmacokinetic parameters: (i) a maximum plasma concentration Cmax of rhein above 5.0 μg/ml; (ii) an area under the concentration time curve AUC0-t or AUC0-∞ of rhein above 35.0 μg·hr/ml; (iii) Tmax of about 3 to 4.5 hours after oral administration to the subject under a fed condition; and (iv) a plasma concentration of rhein above 2.8 μg/ml for at least 4 hours.

20. The formulation according to claim 16, which is a once-daily controlled-release formulation.
